(19) United States
(12) Patent Application Publication
(10) Pub. No.: US 2012/0259413 A1
(43) Pub. Date: Oct. 11, 2012

(54) DEVICES, COMPOSITIONS AND METHODS UTILIZING EP4 AND EP2 RECEPTOR AGONISTS FOR PREVENTING, REDUCING OR TREATING CAPSULAR CONTRACTURE

(75) Inventors: Guang-Liang Jiang, Irvine, CA (US); Wha Bin Im, Irvine, CA (US); Gurpreet Ahluwalia, Orange, CA (US); Frederick C. Beddingfield, Pacific Palisades, CA (US); Larry A. Wheeler, Irvine, CA (US); Scott M. Whitcup, Laguna Hills, CA (US)

(73) Assignee: ALLERGAN, INC., Irvine, CA (US)

(21) Appl. No.: 13/193,774

(22) Filed: Jul. 29, 2011

Related U.S. Application Data
(60) Provisional application No. 61/472,878, filed on Apr. 7, 2011.
(60) Provisional application No. 61/474,195, filed on Apr. 11, 2011.

(51) Int. Cl. A61F 2/12; A61M 35/00; A61P 43/00; A61K 31/381; A61K 31/4015

(52) U.S. Cl. 623/8; 514/443; 514/422; 604/304

(57) ABSTRACT

Provided are devices, compositions and methods utilizing EP₄ and EP₂ receptor agonists for preventing, reducing, or treating capsular contracture occurring in response to the implantation of breast prostheses.

### CROSS-REFERENCE TO RELATED APPLICATIONS

This application claims priority to U.S. Patent Application No. 61/472,878, filed Apr. 7, 2011, and U.S. Patent Application No. 61/474,195, filed Apr. 11, 2011, the entire disclosure of each of which is incorporated herein by this reference.

### FIELD OF INVENTION

The present invention pertains to devices, compositions and methods for treating capsular contracture. More particularly, the present invention relates to devices, compositions and methods for treating capsular contracture using EP4 and EP2 receptor agonists.

### BACKGROUND

Capsular contracture is a common complication that arises following mammoplasty, reconstructive or cosmetic breast surgery to alter the size or shape of the breasts involving the implantation of breast prostheses. Capsular contracture involves the body's formation of a capsule of tissue, primarily collagen fibers (which is scar tissue), around and in response to an implanted object, such as a breast prosthesis. The tightening of the capsule of tissue around a breast prosthesis can be uncomfortable or even extremely painful, and can cause distortion of the appearance of the augmented or reconstructed breast.

Prostaglandins are compounds derived from arachidonic acid which have important functions in the human body. Prostaglandin E₂ (PGE₂), is a well-known type of prostaglandin. Prostanoid receptors are designated according to their endogenous prostaglandin (PG) ligands. For example, the four subtypes of the EP receptor, EP₁₋₄ receptors, have PGE₂ as their endogenous PG ligand. EP₁₋₄ receptors are all part of the G-protein-coupled receptor family. The EP₂ and EP₄ receptors are similar in that both are coupled via Gαₛ to induce elevations in intracellular cAMP and are often co-located on the same cell or tissue types. Both receptors play important regulatory roles in many physiological processes, such as fertility and inflammation.

The current invention presents a solution to capsular contracture by providing devices, compositions and methods utilizing EP₄ and EP₂ receptor agonists for preventing, reducing, or treating capsular contracture occurring in response to the implantation of breast prostheses.

### SUMMARY OF THE INVENTION

The present invention relates to pharmaceutical compositions for preventing, reducing, or treating capsular contracture, the composition comprising a therapeutically effective amount of a compound of Formula I or a pharmaceutically acceptable salt thereof, Compound I or a pharmaceutically acceptable salt thereof, Compound II or a pharmaceutically acceptable salt thereof, or a combination thereof. The compound(s) may be present alone or in combination with one or more pharmaceutically acceptable excipients.

The present invention further relates to methods for preventing and treating capsular contracture occurring in response to the implantation of breast prostheses, the method comprising administering a composition comprising a therapeutic compound selected from the group consisting of EP4 receptor agonists, EP2 receptor agonists, or a combination thereof, to a patient undergoing mammoplasty at various skin sites and at different times, in an amount from about 0.0001 to about 2 mg/kg/day.

The present invention even further relates to devices for preventing and treating capsular contracture occurring in response to the implantation of breast prostheses, the devices include tissue expander, permanent breast prosthesis, medical dressing, drug delivery device, for example, in the form of a drug delivery bra and/or drug delivery bra cushions, which release a composition comprising a therapeutic compound selected from the group consisting of EP4 receptor agonists, EP2 receptor agonists, or a combination thereof.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1A is a Western Blot image of myofibroblast marker expression under the treatment of EP₄ or EP₂ receptor agonists in combination with TGF-β1 in cultured human skin fibroblasts.

FIGS. 1 and 2 show the beneficial effect of the Compound 1 treatment on the healing of the epidermal layer on incisional skin wounds in rats.

FIG. 3 shows the contrast between Compound 1-treated skin and vehicle (composition without Compound 1)-treated skin on epidermis thickness (the ratio of epidermis thickness at wound sites over nearby normal epidermis were shown).

FIG. 4 shows that Compound I significantly reduced polymorphonuclear cell infiltration at wound sites.

FIGS. 5, 6A and 6B show that Compound I treatment significantly reduced the width in the middle and bottom parts of scars, but displayed only a tendency to decrease scar width at the superficial region at 14 days post-surgery; Compound I-treated animals had smaller and softer skin scar, and significantly slimmer appearances than vehicle-treated animals (FIGS. 5 and 6B).

FIGS. 7A and 7B show that the widths of the abnormal structured dermis regions in wounds at 14 days post-surgery (processed for Picrosirius red staining and Masson trichrome collagen staining, respectivley) were significantly smaller in both TGF-β3 and Compound I treated groups than that of vehicle treated group (p<0.01-0.05).

FIG. 8 shows that the size of residual scar regions at 70 days post-surgery (processed for Masson trichrome staining) was remarkably smaller in both TGF-β3 and Compound I-treated groups than that of vehicle-treated group. The effect of Compound I was more noticeable than TGF-β3 (p<0.01-0.05)

FIGS. 9A and 9B show that EP4 receptor agonist application on incisional skin wound affects bFGF and VEGF expression.

### DETAILED DESCRIPTION

The current invention provides devices, compositions and methods utilizing EP₄ and EP₂ receptor agonists for preventing, reducing, or treating capsular contracture occurring in response to the implantation of breast prostheses.

The following are examples of compounds useful for practicing the current invention:

Compounds of Formula (I) or pharmaceutically acceptable salts thereof:

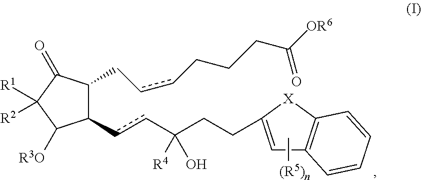

wherein each dashed line represents the presence or absence of a double bond;

R¹, R², R³ and R⁴ are each independently selected from H and C₁-C₆ alkyl;

R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O.

In certain embodiments, R⁴ is H, R³ is H, and X is S.

In another embodiment, R¹ and R² are CH₃.

In a further embodiment, R⁵ is Cl.

### DEFINITIONS

“Alkyl” refers to a monovalent linear or branched hydrocarbon radical having 1 to 6 carbon atoms. Examples include, but are not limited to, methyl, ethyl, propyl (e.g., 1-propyl, isopropyl), butyl (e.g., 1-butyl, isobutyl, sec-butyl, tert-butyl), pentyl (e.g., 1-pentyl, neopentyl), and hexyl (e.g., 3-hexyl).

“Alkenyl” refers to a monovalent linear or branched hydrocarbon radical having 2 to 6 carbon atoms and one or more double bonds. Examples include, but are not limited to, ethenyl, propenyl, and butenyl.

“Halogen” refers to bromo, chloro, fluoro, or iodo.

“Pharmaceutically acceptable salt” refers to any salt of compounds claimed in this application that possesses the biological effectiveness to the said compounds and are not toxic or otherwise harmful for pharmaceutical use; these salts may be derived from organic and inorganic counter ions which are well known in the art.

In yet another embodiment, the compound is Compound I or pharmaceutically acceptable salts thereof:

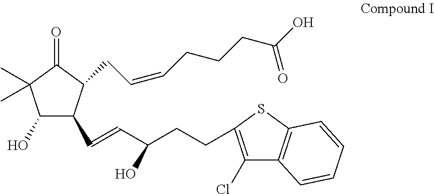

Compound I is an EP₄ receptor agonist as revealed by radioligand binding assays and cAMP assays:

Binding Ki: 6.7±0.7 nM, and EC₅₀ for increased cAMP production and FLIPR

Ca²⁺ signal (hEP4/Gqs): 0.25±0.03 and 0.11±0.05 nM.

The following compound, Compound II, or pharmaceutically acceptable salts thereof, would also be useful for practicing the current invention:

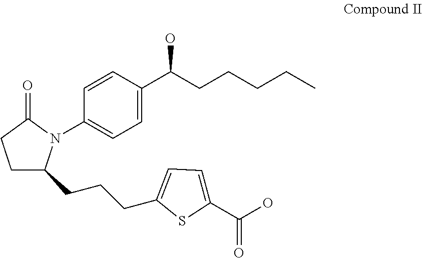

Compound II is an EP₂ receptor agonist as revealed by radioligand binding assays and cAMP assays:

Binding Ki: 21 nM; cAMP enhancement EC₅₀: 0.19 nM; FLIPR Ca2+ signal EC₅₀: 6.7 nM.

General Protocol for Radioligand Binding Assays and cAMP assays:

Radioligand binding studies on plasma membrane fractions prepared from cells** are performed as follows. Cells washed with TME buffer are scraped from the bottom of the flasks and homogenized for 30 sec using a Brinkman PT 10/35 polytron. TME buffer is added as necessary to achieve a 40 ml volume in the centrifuge tubes. TME is comprised of 50 mM TRIS base, 10 mM MgCl₂, 1 mM EDTA; pH 7.4 is achieved by adding 1 N HCl. The cell homogenate is centrifuged at 19,000 rpm for 20-25 min at 4° C. using a Beckman Ti-60 or Tt-70 rotor. The pellet is then re-suspended in TME buffer to provide a final protein concentration of 1 mg/ml, as determined by Bio-Rad assay. Radioligand binding assays are performed in a 100 μl or 200 μl volume.

The binding of [³H] PGE₂ (specific activity 165 Ci/mmol) is determined in duplicate and in at least 3 separate experiments. Incubations are for 60 min at 25° C. and are terminated by the addition of 4 ml of ice-cold 50 mM TRIS-HCl followed by rapid filtration through Whatman GF/B filters and three additional 4 ml washes in a cell harvester (Brandel). Competition studies are performed using a final concentration of 2.5 or 5 nM [³H] PGE₂ and non-specific binding is determined with 10⁻⁵ M unlabelled PGE₂.

For all radioligand binding studies, the criteria for inclusion are >50% specific binding and between 500 and 1000 displaceable counts or better.

cAMP assay was carried out using AlphaScreen cAMP assay kits (PerkinElmer, Boston, Mass.) following manufacturer instructions. Intracellular Ca²⁺ was monitored using a FLIPR Tetra system and assay kits from Molecular Devices following manufacturer instructions. All assays were carried out in HEK-293 cells heterologously and stably expressing each of the eight human recombinant prostanoid receptors. For Ca²⁺ signals, hEP2, hEP4, hDP were co-expressed with a chimeric G protein, Gqs, which converts Gs signal to Gq Ca²⁺ signal, and hEP3 with a chimeric G protein, Gqi. Each receptor-selective agonist induced Ca²⁺ signals with sub-nanomolar or nanomolar EC₅₀ values. Subtype-selective compounds used here are PGE₂ for EP1, EP2, EP3 and EP4; BW245C for DP; 17-phenyl PGF2α for FP, carbacyclin for IP and U-46619 for TP.

**Cells:

### Human Recombinant EP₁, EP₂, EP₃, EP₄, FP, TP, IP and DP Receptors: Stable Transfectants

Plasmids encoding the human EP₁, EP₂, EP₃, EP₄, FP, TP, IP and DP receptors are prepared by cloning the respective coding sequences into the eukaryotic expression vector pCEP₄ (Invitrogen). The pCEP₄ vector contains an Epstein Barr virus (EBV) origin of replication, which permits episomal replication in primate cell lines expressing EBV nuclear antigen (EBNA-1). It also contains a hygromycin resistance gene that is used for eukaryotic selection. The cells employed for stable transfection are human embryonic kidney cells (HEK-293) that are transfected with and express the EBNA-1 protein. These HEK-293-EBNA cells (Invitrogen) are grown in medium containing Geneticin (G418) to maintain expression of the EBNA-1 protein. HEK-293 cells are grown in DMEM with 10% fetal bovine serum (FBS), 250 μg ml⁻¹ G418 (Life Technologies) and 200 μg ml⁻¹ gentamicin or penicillin/streptomycin. Selection of stable transfectants is achieved with 200 μg ml⁻¹ hygromycin, the optimal concentration being determined by previous hygromycin kill curve studies.

For transfection, the cells are grown to 50-60% confluency on 10 cm plates. The plasmid pCEP₄ incorporating cDNA inserts for the respective human prostanoid receptor (20 μg) is added to 500 μl of 250 mM CaCl₂. HEPES buffered saline×2 (2×HBS, 280 mM NaCl, 20 mM HEPES acid, 1.5 mM Na₂ HPO₄, pH 7.05-7.12) is then added drop-wise to a total of 500 μl, with continuous vortexing at room temperature. After 30 min, 9 ml DMEM are added to the mixture. The DNA/DMEM/calcium phosphate mixture is then added to the cells, which is previously rinsed with 10 ml PBS. The cells are then incubated for 5 hr at 37° C. in humidified 95% air/5% CO₂. The calcium phosphate solution is then removed and the cells are treated with 10% glycerol in DMEM for 2 min. The glycerol solution is then replaced by DMEM with 10% FBS. The cells are incubated overnight and the medium is replaced by DMEM/10% FBS containing 250 μg ml⁻¹ G418 and penicillin/streptomycin. The following day hygromycin B is added to a final concentration of 200 μg ml⁻¹.

Ten days after transfection, hygromycin B resistant clones are individually selected and transferred to a separate well on a 24 well plate. At confluence each clone is transferred to one well of a 6 well plate, and then expanded in a 10 cm dish. Cells are maintained under continuous hygromycin selection until use.

Methods of preparing the disclosed compounds and additional compounds suitable for use in the methods disclosed herein, can be found in, e.g., Donde, et el., 10,10-Dialkyl Prostanoic Acid Derivatives as Agents for Lowering Intraocular Pressure, U.S. Pat. No. 6,875,787; Donde, et el., 10,10-Dialkyl Prostanoic Acid Derivatives as Agents for Lowering Intraocular Pressure, U.S. Patent Publication 2004/0235958; Donde, et al., Treatment of Inflammatory Bowel Disease, U.S. Patent Publication 2005/0164992, each of which is hereby incorporated by reference in its entirety.

### Lab Results

EP4 and EP2 Receptor Agonists Inhibit TGF-β1-Induced Myofibroblasts Formation

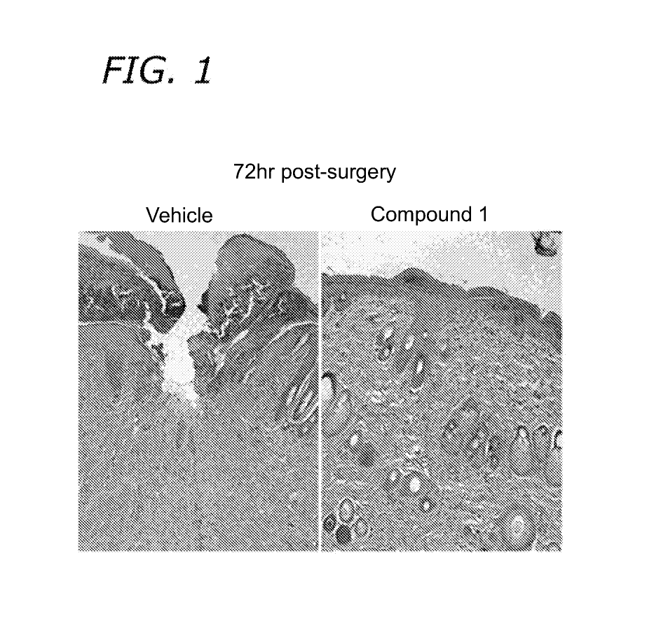

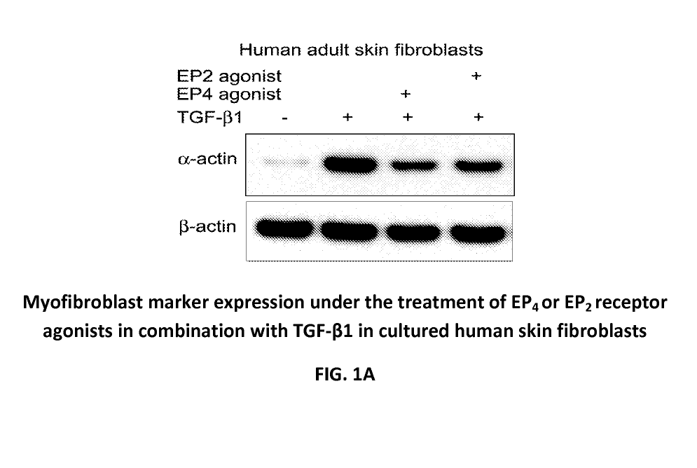

Adult skin fibroblasts were derived from normal skin of a 61-year old Caucasian female, purchased from ATCC(CRL-7346). Cells were cultured in DMEM medium supplemented with 10% fetal bovine serum and 1% streptomycin and penicillin in incubators at 37° C. and 5% CO₂. Cells were seeded in 10 cm dishes at 1×10⁶ cells/dish. When the cells became 80% confluent, they were cultured in serum-free medium for 48 hrs after washing away residual serum with PBS. Vehicle, Compound I (EP₄ receptor agonist), or Compound II (EP₂ receptor agonist), together with human recombinant TGF-β1 (2 ng/ml), were added to culture medium at 0, 10 or 100 nM final concentration, respectively. Compound I or 2 was first dissolved in DMSO, the final DMSO concentration was 0.1%. Cell lysates were collected at 72 hours after treatments, respectively. Proteins were quantified and resolved on 4-10% SDS-PAGE. Then the proteins were transferred to membrane by electrophoresis. The membranes were blocked with mouse-anti-alpha smooth muscle actin (α-SMA) antibody, and a second antibody against mouse-IgG conjugated with AP (purchased from Signal Transduction). Shown in FIG. 1A is a representative image of Western Blot myofibroblast marker expression under the treatment of EP₄ or EP₂ receptor agonists in combination with TGF-β1 in cultured human skin fibroblasts. TGF-β1 treatment significantly up-regulated the expression of α-SMA, which was dramatically ameliorated by co-treatment with the EP₄ or EP₂ receptor agonist.

EP₄ and EP₂ receptor agonists are known to enhance intracellular cAMP level potently, and cAMP has been reported to block the expression of TGF-β1-induced connective tissue growth factor. (See Kothapalli, D., Hayashi, N., Grotendorst, G. R., “Inhibition of TGF-beta-stimulated CTGF gene expression and anchorage-independent growth by cAMP identifies a CTGF-dependent restriction point in the cell cycle,” FASEB J., 12(12): 1151-61 (1998)). CTGF is a strong inducer for fibrosis. Therefore, EP₄ and EP₂ receptor agonists inhibit TGF-β1-induced connective tissue growth factor expression.

### Incisional Skin Wound Study

The Effect of Compound I (EP₄ Receptor Agonist) on Wound Healing

Sprague-Dawley rats weighing 180-200 grams were anesthetized with isoflourane. After shaving, a 2-cm long incision was made, reaching the deep fascia on the back skin of rats under sterile conditions. The wounds were immediately closed with 4-0 sutures. A 14 day pilot study was carried out. The animals were topically treated with vehicle or Compound 1 at 0.004% twice daily. The vehicle contained ethanol 30%, propylene glycol 12%, dipropylene glycol 5%, benzyl alcohol 5%, glycerol 3% and normal saline 45%. The wound was photographed daily; biopsy was performed at 2, 3, 7 and 14 days post-surgery for histopathology and molecular biology analysis.

A similar skin wound study was also performed comparing the effects of Compound I and TGF-β3. In this study, intradermal injections of Compound I at 0.004%, TGF-β3 at 100 ng/200 μl or vehicle were given right before closing the wounds. Afterward, TGF-β3 was injected two more times, on day 1 and 2, and Compound I and vehicle were topically applied twice a day for the duration of the study. The vehicle was PBS with 0.1% BSA and 4 mM HCl in a total volume of 200 μl for injection. Skin wounds were imaged on day 3, 7, 14, 35 and 70.

The wound tissue was biopsied for histopathology on day 3, 14 and 70. To observe the skin wound, paraffin-embedded wound sections were made. Regular H&E staining was carried out in comparison with Masson trichrome and/or Picosirus red to visualize the collagen fibers. To monitor myofibroblasts in skin wound, the sections were immunohistochemically stained to identify alpha-smooth muscle actin. To assess wound appearance, all the scar photos were mixed together by the end of each study. The scar severity was scored on a scale of 0 to 10, with 0 being invisible, 1 the minimal and 10 the worst. Each scar was divided into 4 regions, separated by suture sites; each quarter was scored independently; the mean of the 4 part scores was recorded as the gross score of each wound.

On day 3, 80% of skin wound samples treated with Compound I showed closed epidermis filled with keratinocytes, while only 33% of vehicle treated wounds had closed epidermis (FIGS. 1 and 2). The overall size of epidermal defects was two times larger for vehicle-treated wounds as compared with that of Compound I treated wounds (FIGS. 1 and 2). This demonstrates a beneficial effect of the Compound I treatment on the healing of the epidermal layer.

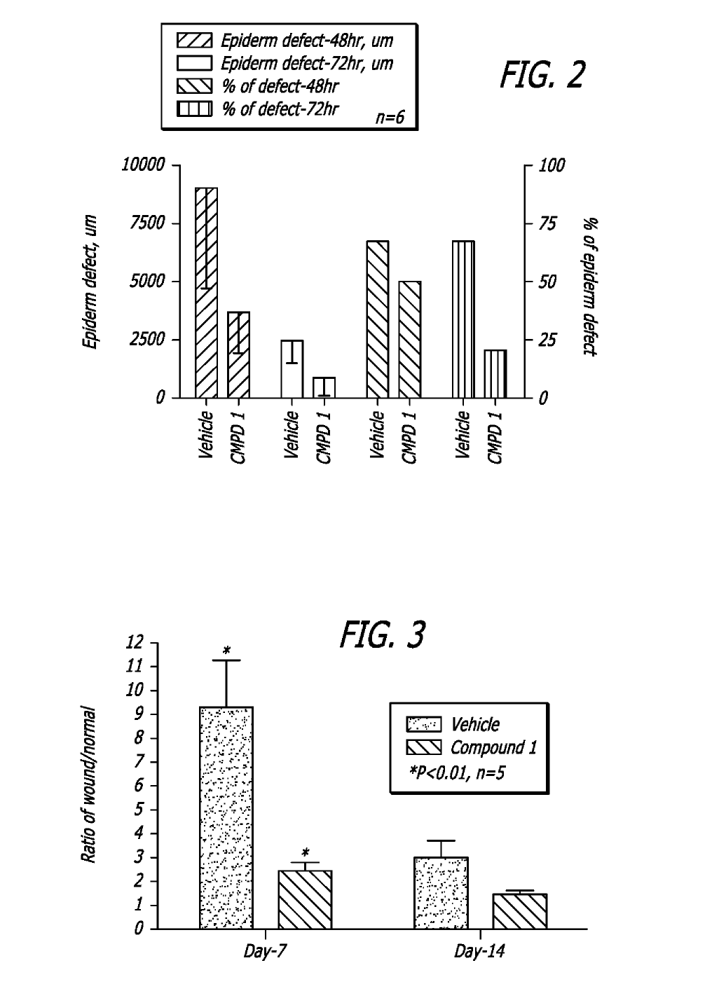

On 7 days post-skin incision, the epidermal layer of Compound I treated skin not only had a thickness close to the nearby normal epidermis, but also had epidermal wrinkle resembling normal elastic skin structure. In contrast, the vehicle-treated skin had epidermal hyperplasia with a thickness of 3 times more than the Compound I treated epidermis (FIG. 3).

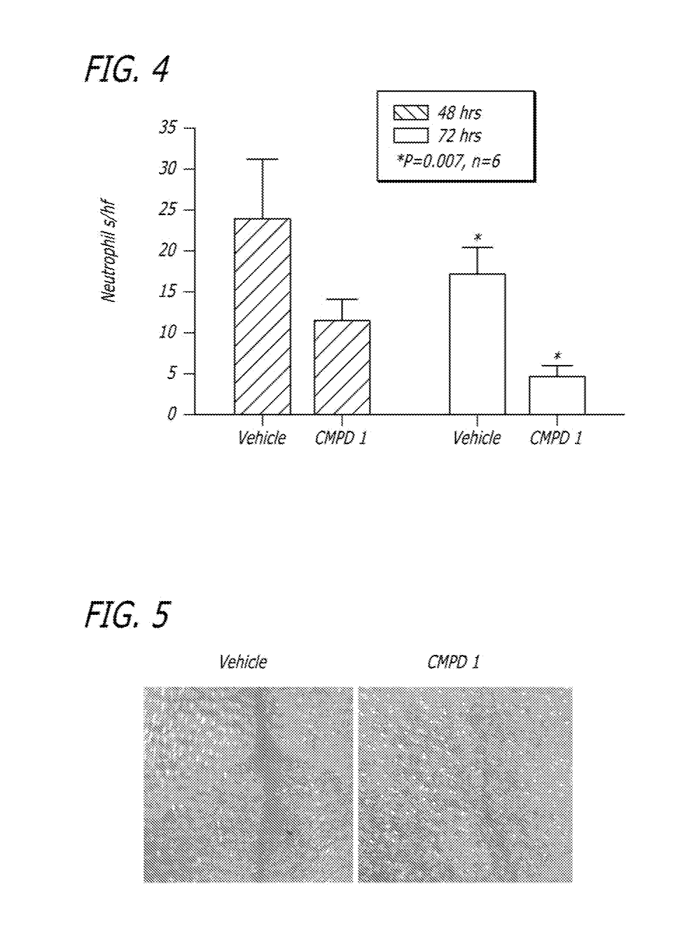

Neutrophils are recruited to injury sites as the first innate immune response. Their lysis and release of chemokines attract other inflammation cells and amplify inflammatory processes. Neutrophil infiltration was monitored on sectioning tissue on days 2 and 3. Compound I significantly reduced polymorphonuclear cell infiltration at wound sites (FIG. 4).

Myofibroblasts were identified by immunohistochemical staining of alpha-smooth muscle actin (α-SMA) on sections from day 2 to day 14 post-surgery. Both staining and assessment were conducted by personnels blinded to the treatments. Strong α-SMA signals were localized at the cytoplasm of large cells, and such α-SMA-positive cells were mainly distributed along the granulation tissue at the dermis layer at wound sites. Abundant myofibroblasts were observed on day 3 samples, which indicated their proliferation during adult scar wound healing. Compound I treatment reduced the number of myofibroblasts (25.8±7.45/3 sections) as compared to vehicle control (38±6.15/3 sections).

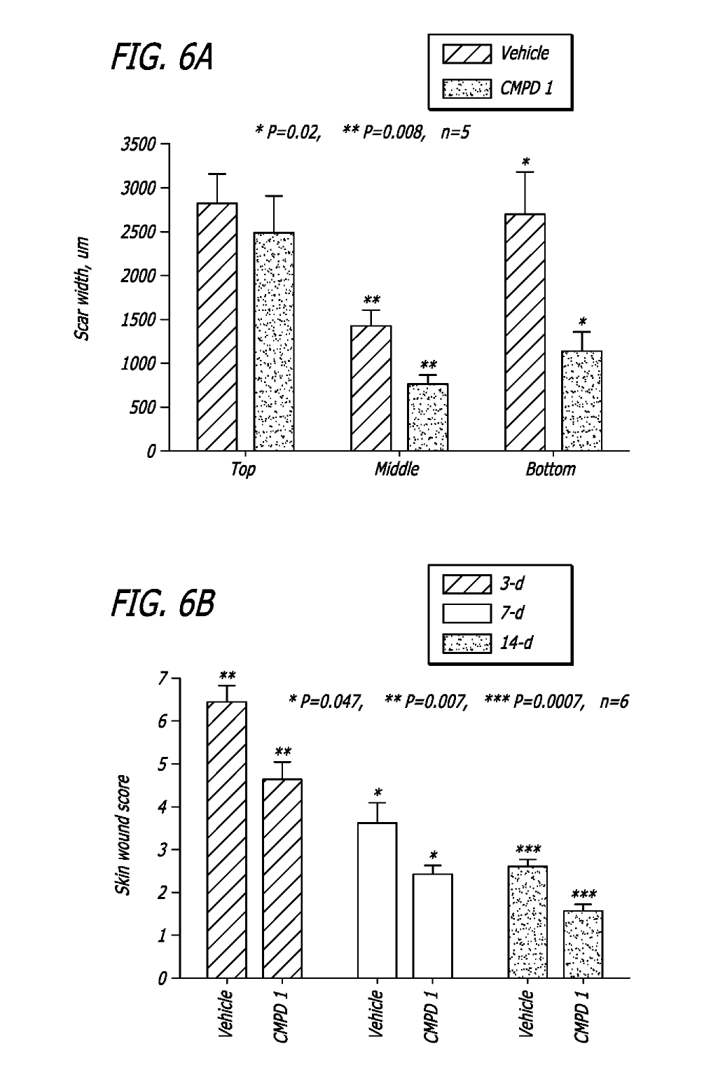

Biopsy samples of skin wound tissues were analyzed at 7 and 14 days post-surgery. Tissue samples about 1 mm wide were taken from both sides of the wound. Sections from day 14 were stained for collagen fibers by Masson Trichrome. The scar sites contained fine, short, lightly stained collagen fibers, positioning somewhat parallel to the epidermis, but generally in unstructured fashion. In normal dermis, the collagen fibers were thick, long, deeply stained, and clearly organized in a basket-weave mode, which appears to be central to the elasticity and tensile of normal skin. The width of the abnormal fiber belt was measured at the surface, the middle and the bottom of scars. Compound I treatment significantly reduced the width in the middle and bottom parts of scars, but displayed only a tendency to decrease scar width at the superficial region (FIGS. 5 and 6 A and B). Grossly, Compound I treated animals had smaller and softer skin scar, and significantly slimmer appearances than vehicle-treated animals (FIGS. 5 and 6 A and B).

Since TGF-β3 is a leading treatment for wounds, reportedly reducing skin scar in both animals and human, the effect of Compound I and TGF-β3 were compared. Here, the focus was on three temporal phases of wound healing and scar formation: inflammation on day 3, overall wound healing on day 14, and scar remodeling on day 70. Neutrophil infiltration, a hall mark of inflammation, was easily detectable 3 days post-surgery. The number of neutrophils was counted in three sections of H&E stained tissues; they were 60.6±30, 53.8±17 or 31.4±8 for vehicle, TGF-β3 or Compound I treated groups, respectively. The trend of suppressed neutrophil infiltration by Compound I was apparent, albeit not statistically significant due to small samples (n=5), and is consistent with our previous observation.

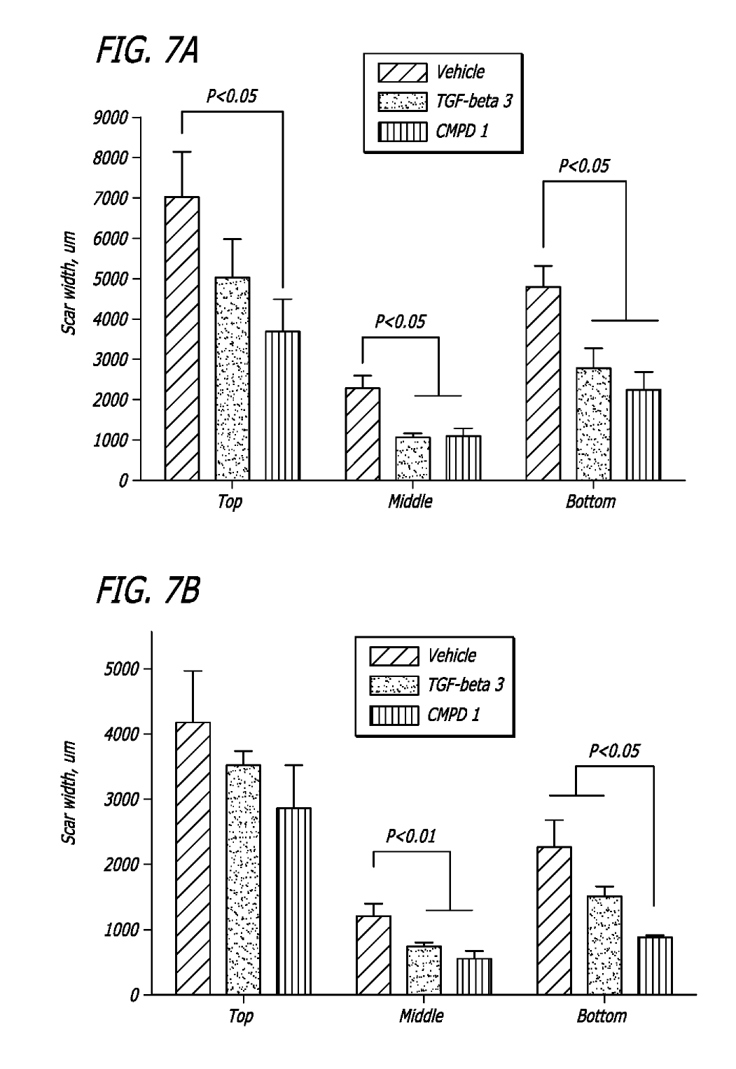

At day 14, wounded skin tissue was processed for both Picrosirius red and Masson trichrome collagen staining. For Picrosirius-stained tissues under polarized light, type I collagen fibril appears in yellow color and type III collagen in green. Vehicle-treated wounds showed some green, fine fibrils in gaps, but not yellow, large fibril bundles. The TGF-β3-treatment also had some green fibers at the bottom of the wounds, but Compound 1 treatment showed large yellow-stained collagen bundles almost crossing over the entire wound sites, with little green-stained type III collagen (FIGS. 7A and B). Also the gap width in-between the normal fibrils was significantly narrower in both TGF-β3-treated and Compound I treated groups than that of vehicle-treated group (p<0.05, FIGS. 7A and B). This indicated that Compound 1 treatment not only reduced the abnormal structured gap but also diminished immature type III collagen at wound sites.

Different sections of the same wounds were also processed for Masson trichrome collagen staining. Collagen at nearby normal skin was stained as dark-blue, thick bundle oriented in a basket-weave reticular pattern. A distinctive region at the wound site was stained as fine, thin collagen fibers in parallel to epidermis. The demarcation between normal and abnormal region was quite clear. The widths of the abnormal structured dermis regions were significantly smaller in both TGF-β3 and Compound I treated groups than that of vehicle treated group (p<0.01-0.05, FIGS. 7A and B).

Skin wound at a later phase undergoes remodeling. At 70 days post-surgery, wound sites showed different features of collagen staining from those seen 14 days post surgery. On Picrosirius red stained sections, wound gaps in vehicle-treated group were now filled by dense, red, fine fibers in a parallel orientation. Such abnormal regions were largely absent in both TGF-β3 and Compound I treated groups. Instead, more abundant yellow, thick bundles of collagen fibers in a basket-weave pattern was observed than the vehicle-treated skin.

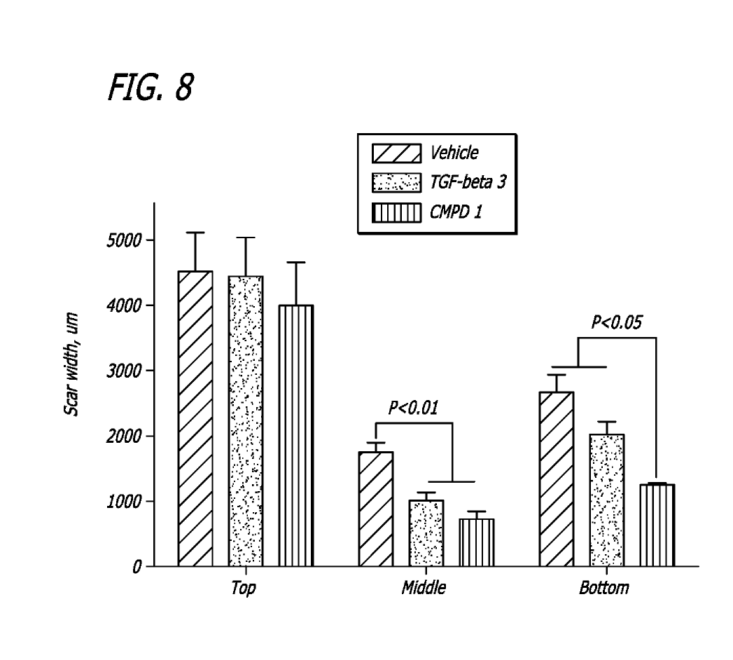

Masson trichrome staining also revealed temporal changes in scar remodeling. On day 70, the scar regions were filled with fine, thin collagen fibers more densely than on day 14. The demarcation between normal and abnormal region became much more distinctive than on day 14. The size of residual scar regions was remarkably smaller in both TGF-β3 and Compound I-treated groups than that of vehicle-treated group. The effect of Compound I was more noticeable than TGF-β3 (p<0.01-0.05, FIG. 8).

Also the macroscopic surface appearance of wound sites was monitored 70 days post-surgery. In the vehicle treatment, wound sites were replaced with white, shiny, firm, slightly raised scars. The TGF-β3 treatment still showed traces of wounds, although much improved over the vehicle treatment. With the Compound I treatment, wound sites were not even detectable, if not for two indication markings on the tissue.

### Compound I (EP₄ Receptor Agonist) Enhances Expression of VEGF and bFGF at Wound Sites

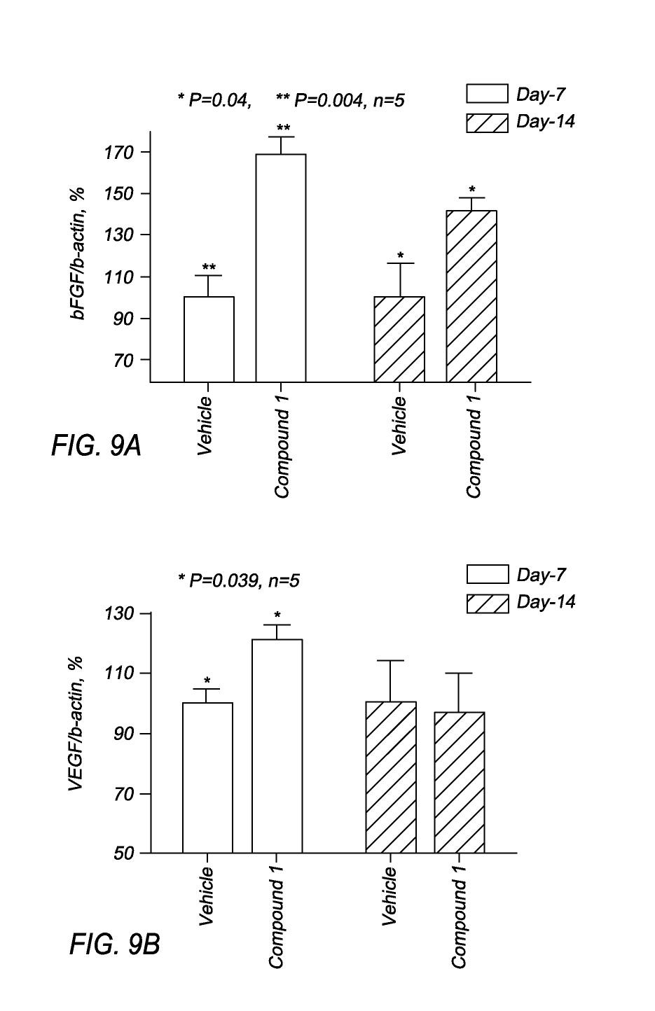

2 cm long incisional full thickness skin wounds were made on the back of Sprague Dawley rats. Wounds were treated with vehicle or EP₄ agonist (compound I). The wound tissue was biopsied at day-7 or day-14 post-surgery. The samples were homogenized in protein extraction buffer after being frozen in liquid nitrogen. The protein concentrations were measured. Then the samples were loaded into 4-10% SDS-Pages to resolve the proteins. After electrophoresis, the proteins were transferred onto nitrocellular membranes. The membranes were blocked with anti-VEGF, anti-bFGF or anti-beta-actin, respectively. The beta actin served as internal control for comparable loading amounts. As shown in FIGS. 9A and 9B, EP₄ agonist treatment enhanced bFGF expression by up to 60% at both day-7 and day-14 time points. It is reported that bFGF prevents scar formation and reduces hypertrophic and burn-induced skin scars. VEGF expression was boosted by 25% transiently at day-7, which may contribute to angiogenesis at wound site and facilitate scar-free healing.

During and post-breast implantation, the tissues surrounding breast implants begin a healing response, which includes disruption of clotted platelets and release of pro-inflammatory cytokines; aggregation of neutrophils, monocytes and lymphocytes; fibroblasts proliferation and transformation into myofibroblasts; deposition of extracellular matrix and fibrosis formation, as well as vasculature regeneration to improve compromised blood supply due to surgical injuries (See Tan, K. T., et al., “Tumor necrosis factor-α expression is associated with increased severity of periprosthetic breast capsular contracture,” Eur Surg Res., 45 (3-4):327 (2010); Moreira, M., et al., “The effect of liposome-delivered prednisolone on collagen density, myofibroblasts, and fibrous capsulae thickness around silicone breast implants in rats,” Wound Repair Regen. 2010, 18(4):417.) Due to the living body's natural response to foreign objects implanted in the body, inflammation surrounding breast implants stays active; and the deposition of extracellular matrix by myofibroblasts and fibroblasts goes on and on and results in a dense fibrosis capsule around the implant. Eventually, this fibrotic capsule may contract and deform the implant. The proposed therapeutics, EP₄ and EP₂ receptor agonists, may disrupt the pathogenesis at several steps based on their mechanism of action on skin wound healing, such as minimizing the inflammatory cytokine effects, suppressing myofibroblast formation and reducing fibrosis or improving local circulation through angiogenesis. It is known locating breast implants at sites with good circulation results in lower contracture incidence.

The present invention relates to pharmaceutical compositions for preventing, reducing, or treating capsular contracture, the compositions comprising a therapeutically effective amount of a compound of Formula (I), Compound I, Compound II, or combinations thereof, said compound being present alone or in combination with one or more pharmaceutically acceptable excipients.

An “acceptable” excipient is one that is compatible with the active ingredient of the composition and not harmful to the person being administered the pharmaceutical composition.

As used herein, the term “therapeutically effective amount” means the amount of the pharmaceutical or cosmetic composition that will elicit the biological, medical, or cosmetic response of a subject in need thereof that is being sought by the researcher, veterinarian, medical doctor or other clinician. Effective amounts of the compound may be determined by one of ordinary skill in the art, but will vary depending on various factors, such as the frequency of application. For example, an effective amount might be about 0.0001 to about 2 mg/kg/day.

The present invention also relates to devices and methods for preventing, reducing, or treating capsular contracture occurring in response to the implantation of breast prostheses utilizing EP₄ and EP₂ receptor agonists.

### EXAMPLES

The following are examples illustrating embodiments of the present invention.

### Example 1

In one aspect, the present invention relates to a method for preventing, reducing, or treating capsular contracture occurring in response to the implantation of breast prostheses, the method comprising administering a composition comprising a therapeutic compound selected from the group consisting of EP₄ receptor agonists, EP₂ receptor agonists, or a combination thereof, to the whole surface of the breasts of a patient undergoing mammoplasty, a surface of the breasts which include the intact sites of incision, or just the intact sites on the breasts that will serve as sites of incision on a patient undergoing mammoplasty, prior to the first incision, in an amount from about 0.0001 mg to about 2 mg/kg/day. The composition containing the therapeutic compound(s) can also be applied to the incision sites as the incisions are made.

Following skin sterilization and draping procedures, the incision sites on an anesthetized patient about to undergo mammoplasty are exposed and ready for the initial incision and the subsequent dissection for pocket development. A composition containing an EP₄ receptor (such as Compound I) agonist, EP₂ receptor agonist (such as Compound II), or a combination thereof (the composition may be in the form of a liquid, gel, lotion, cream or the like) will be applied to the intact sites of incision, or a surface of the breasts which include the intact sites of incision, or the whole surface of the breasts, prior to the first incision. The locations for incision depend on the particular incision technique chosen. Examples of incision techniques include the periareolar, inframammary, transumbilical, and transaxillary incision options. The periareolar incision location, for example, is at the junction between the pigmented skin of the areola and the lighter skin of the breast (e.g., the inferior border of the areola). The incisions can be made about 30 minutes after the application of the composition containing the therapeutic compound(s).

Moreover, during the making of an incision, surgical dressing dipped in a composition containing an EP₄ receptor (such as Compound I) agonist, EP₂ receptor agonist (such as Compound II), or a combination thereof (the composition may be in the form of a liquid, gel, lotion, cream or the like) can be used to apply the therapeutic compound(s) to the incision site when the surgical dressing is used to clear blood from the site. Applying the therapeutic compound(s) prior and/or during the making of any incision activates EP2/EP4-mediated signaling ahead of TGF-β1's release from wounded tissue, which prevents TGF-β1-induced scar-forming cascades.

### Example 2

In one aspect, the present invention relates to a method for preventing, reducing, or treating capsular contracture occurring in response to the implantation of breast prostheses, the method comprising administering a composition comprising a therapeutic compound selected from the group consisting of EP₄ receptor agonists, EP₂ receptor agonists, or a combination thereof, to dissection sites on the breast during or immediately following the dissection for pocket development, in an amount from about 0.0001 mg to about 2 mg/kg/day.

Following making of the initial incision, there must be dissection to develop the pocket in which an implant will be placed. Just as for making of the incision, there are different dissection techniques available for pocket development. For example, following a periareolar incision, the transparenchymal technique can be used in which dissection can proceed directly down through the breast to the pectoralis major muscle, then a subglandular, subfascial, or subpectoral pocket can be created. The periaparenchymal technique is another option, in which dissection proceeds inferiorly around the lower pole of the breast at the level of the breast capsule until the inframammary fold is reached, then superiorly up and under the breast to create the desired pocket. (Hammond, D. C., “The Periareolar Approach to Breast Augmentation,” Clin Plastic Surg 36 (2009) 45-48). Blunt dissection would be required if remote-access incision techniques, such as the transumbilical technique, is used to create the initial incision.

Instruments used for separating of, for example, muscular fibers to open up the submuscular space, can be coated with a composition (the composition may be in the form of a liquid, gel, lotion, cream or the like) comprising a therapeutic compound selected from the group consisting of EP₄ receptor agonists, EP₂ receptor agonists, or a combination thereof, such that the therapeutic compound is applied onto the sites of dissection during dissection. Other ways of administering the therapeutic compound(s) can also be used, such as by applying the composition containing the therapeutic compound(s) using surgical dressing. Administration of the compound(s) immediately after dissection is also desirable. The amount of therapeutic compound(s) applied should be about 0.0001 to about 2 mg/kg/day; the concentration of the therapeutic compound(s) in liquid, gel, lotion, cream, etc., formulations should be about 0.0001% to about 0.01%.

### Example 3

In one aspect, the present invention relates to an implantable prosthesis, for example, a tissue expander for preventing, reducing, or treating capsular contracture occurring in response to the implantation of breast prostheses, the tissue expander comprising an inflatable envelope, a fillable cavity enclosed by the envelope, and a structure coupled to the envelope and effective to release a composition comprising a therapeutic compound selected from the group consisting of EP₄ receptor agonists, EP₂ receptor agonists, or a combination thereof, in an amount from about 0.0001 mg to about 2 mg/kg/day, for the duration of the tissue expander's implantation in the patient, which typically lasts between two and six months.

The structure effective to release the composition may be in the form of a mechanism, such as one or more osmotic pumps known in the medical device art and discussed in greater detail hereinafter. Other suitable structures effective to release a composition include coatings on the envelope, or any other suitable mechanism known in the art which will be capable of releasing the desired composition into the patient in conjunction with the implantation of the prosthesis.

In another aspect, the present invention relates to an implantable tissue expander for preventing, reducing, or treating capsular contracture occurring in response to the implantation of breast prostheses, the tissue expander comprising an inflatable envelope, a fillable cavity enclosed by the envelope, and silk fibroin hydrogel coating which releases a composition comprising a therapeutic compound selected from the group consisting of EP₄ receptor agonists, EP₂ receptor agonists, or a combination thereof, in an amount from about 0.0001 mg to about 2 mg/kg/day, for the duration of the tissue expander's implantation in the patient, which typically lasts between two and six months.

Soft tissue, such as skin and muscle, can expand to accommodate the growth of underlying structures. For example, abdominal skin and muscle expand during pregnancy. Soft tissue can also be gradually expanded using a device known as a tissue expander. Tissue expanders are used, for example, to promote tissue growth to make room or develop a pocket of a desired size and shape for the eventual insertion of a permanent prosthesis. A tissue expander is typically constructed out of penetrable, self-sealing and stretchable material, such as an elastomeric material like silicone.

A tissue expander is first subcutaneously implanted in a contracted state at a location, then gradually enlarged by the injection of fluid, such as saline, into a cavity or chamber inside the expander. As the tissue expander expands, so does the skin overlying or covering the tissue expander. A tissue expander is only temporary breast prosthesis. Once the skin has expanded to a desired capacity or size (sufficient to accommodate the permanent prosthesis that will be inserted), the tissue expander is removed before a permanent prosthesis is inserted into the space created by the tissue expander.

After a tissue expander is first implanted subcutaneously, fibrous capsule develops as the wounds created by the surgical procedure used to implant the tissue expander heal, and the fibrous capsule constrains the expansion of the expander. Typically, a tissue expander for developing a pocket for a breast prosthesis is tear-drop-shaped, but the constraint of the fibrous capsule might lead to a more spherical-shaped pocket.

The present invention provides a tissue expander which includes features that prevent, reduce, or treat capsular contracture. In one embodiment, the tissue expander of the current invention features one or more osmotic pumps which release a composition comprising a therapeutic compound selected from the group consisting of EP₄ receptor agonists, EP₂ receptor agonists, or a combination thereof, in an amount from about 0.0001 mg to about 2 mg/kg/day, for the duration of the tissue expander's implantation in the patient, which typically lasts between two and six months. Alternatively, the tissue expander features silk fibroin hydrogel coating that release the therapeutic compounds in the desired amount and for the desired period.

Implantable osmotic pumps for drug delivery are known in the art (see U.S. Pat. No. 5,728,396, U.S. Pat. Appl. 2002/0183722). For example, U.S. Pat. No. 5,728,396 discloses an implantable osmotic pump comprising a drug chamber, a water-swellable agent chamber, a movable piston, and a semipermeable membrane; when fluid from the body enters the water-swellable agent chamber through the semipermeable membrane, the water-swellable agent in the water-swellable agent chamber expands, pushes the piston, which causes drug to be released from the drug-chamber through a diffusion outlet at a substantially constant rate.

Materials, such as hydrogels, for example but not limited to, silk fibroin hydrogels, can also be used as a drug delivery mechanism. Silk refers to a filamentous product secreted by an organism such as a silkworm. Fibroin, the primary structural component of silk, is produced and secreted by the silk glands of the organism as a pair of complementary fibrils called “brins.” As fibroin brins leave the glands, they are coated with sericin, a glue-like substance which binds the brins together. Sericin is often antigenic and may be associated with an adverse tissue reaction when sericin-containing silk is implanted in vivo. Sericin may be substantially (i.e., 4% residual sericin by mass in the final extracted silk) removed through known methods, resulting in virtually sericin-free fibroin. For example, natural silk from the silkworm Bombyx mori may be subjected to sericin extraction, spun into yarns, then used to create a matrix with high tensile strength. Silk fibroin can also be made into silk hydrogel, which comprises a silk protein network fully saturated with water, coupling the molecular resiliency of silk with the biocompatibility of a “wet” material. Silk fibroin hydrogel and methods for manufacture of silk fibroin hydrogels are known (see, e.g., U.S. Pat. Appl. No. 61/170,895 “Silk Fibroin Hydrogel Devices”). Generation of a silk fibroin hydrogel may be accomplished by breaking apart native silk fibroin polymers into its individual monomeric components using a solvent species, replacing the solvent with water, then inducing a combination of inter- and intra-molecular aggregation.

For the present invention, the silk fibroin hydrogel coating is formed with a composition comprising a therapeutic compound selected from the group consisting of EP₄ receptor agonists, EP₂ receptor agonists, or a combination thereof, entrained in or bound to the gel. To control the drug release profile, silk solutions can first be mixed with the composition comprising a therapeutic compound selected from the group consisting of EP₄ receptor agonists, EP₂ receptor agonists, or a combination thereof, then form a hydrogel. The silk fibroin hydrogel can be used as a surface coating of a silk yarn or mesh overlying the tissue expander. The composition comprising a therapeutic compound selected from the group consisting of EP₄ receptor agonists, EP₂ receptor agonists, or a combination thereof would be released from the silk fibroin hydrogel component of the tissue expander at a rate of about 0.0001 to about 2 mg/kg/day for the duration of the tissue expander's implantation in the patient, which typically lasts between two and six months.

### Example 4

In one aspect, the present invention relates to a method for preventing, reducing, or treating capsular contracture occurring in response to the implantation of breast prostheses, the method comprising administering a composition comprising a therapeutic compound selected from the group consisting of EP₄ receptor agonists, EP₂ receptor agonists, or a combination thereof, to sites of incision and dissection made on a breast in order to remove a tissue expander, in an amount from about 0.0001 mg to about 2 mg/kg/day, during and following removal of the tissue expander.

Alternatively, or in addition, the composition containing the therapeutic compounds can be administered to the whole surface area of the breast and/or the breast pocket that the tissue expander created.

### Example 5

In one aspect, the present invention relates to an implantable permanent breast prosthesis for preventing, reducing, or treating capsular contracture occurring in response to the implantation of breast prostheses, the breast prosthesis comprising a shell having an outer surface, an inner surface, an internal lumen which can be filled with a fluid or gel, and one or more osmotic pumps which release a composition comprising a therapeutic compound selected from the group consisting of EP₄ receptor agonists, EP₂ receptor agonists, or a combination thereof, in an amount from about 0.0001 mg to about 2 mg/kg/day, for from 5 days up to one month, six month or one year.

In one aspect, the present invention relates to an implantable permanent breast prosthesis for preventing, reducing, or treating capsular contracture occurring in response to the implantation of breast prostheses, the breast prosthesis comprising a shell having an outer surface, an inner surface, an internal lumen which can be filled with a fluid or gel, and silk fibroin hydrogel coating which release a composition comprising a therapeutic compound selected from the group consisting of EP₄ receptor agonists, EP₂ receptor agonists, or a combination thereof, in an amount from about 0.0001 mg to about 2 mg/kg/day, for from 5 days up to one month, six month or one year.

Osmotic pumps and silk fibroin hydrogel are known and discussed in Example 3.

In another aspect, the present invention relates to a method for preventing, reducing, or treating capsular contracture occurring in response to the implantation of breast prostheses, the method comprising covering a permanent breast prosthesis in a composition comprising a therapeutic compound selected from the group consisting of EP₄ receptor agonists, EP₂ receptor agonists, or a combination thereof, in an amount from about 0.0001 mg to about 2 mg/kg/day, before the breast prosthesis is inserted into a breast cavity. The permanent breast prosthesis can be any such prosthesis that is available in the art.

### Example 6

In one aspect, the present invention relates to medical dressing comprising silk fibroin hydrogel which releases a composition comprising a therapeutic compound selected from the group consisting of EP₄ receptor agonists, EP₂ receptor agonists, or a combination thereof, in an amount from about 0.0001 to about 2 mg/kg/day, for about one to about ten days.

Following mammoplasty, the patient's breasts may be covered up with medical dressing during at least part of the recovery period. Medical dressing containing the therapeutic compounds would be useful for preventing, reducing, or treating capsular contracture occurring in response to the implantation of breast prostheses. Silk fibroin hydrogel used for drug delivery is discussed in Example 3.

In one aspect, the present invention relates to a method for preventing, reducing, or treating capsular contracture occurring in response to the implantation of breast prostheses, the method comprising administering a composition comprising a therapeutic compound selected from the group consisting of EP₄ receptor agonists, EP₂ receptor agonists, or a combination thereof, to sites of incision and dissection on a breast following the implantation of a permanent breast prosthesis into a breast cavity, in an amount from about 0.0001 mg to about 2 mg/kg/day, before the breast prosthesis is inserted into a breast cavity. The permanent breast prosthesis can be any such prosthesis that is available in the art.

For example, the composition comprising a therapeutic compound selected from the group consisting of EP₄ receptor agonists, EP₂ receptor agonists, or a combination thereof may be topically applied to an incision or dissection site before the site is covered with gauze and medical bandage. Medical dressings made out of silk fibroin hydrogel that can release a composition comprising a therapeutic compound selected from the group consisting of EP₄ receptor agonists, EP₂ receptor agonists, or a combination thereof, over time (for example, for about 10 days) at a rate of about 0.0001 to about 2 mg/kg/day may be applied to protect incision and dissection sites following implantation of a permanent breast prosthesis.

### Example 7

In one aspect, the present invention relates to a drug delivery device, for example, a drug delivery bra, for preventing, reducing, or treating capsular contracture occurring in response to the implantation of breast prostheses, the drug delivery bra comprising a front breast panel comprising two breast cups, each cup having a layer of silk fibroin hydrogel which releases a composition comprising a therapeutic compound selected from the group consisting of EP₄ receptor agonists, EP₂ receptor agonists, or a combination thereof, in an amount from about 0.0001 mg to about 2 mg/kg/day; a back panel; side panels, each connected to both the front panel and the back panel; shoulder straps and fasteners.

Post-surgery, mammoplasty patients are typically instructed to wear a support bra. The drug delivery bra of the present invention contains a layer of drug-releasing silk fibroin hydrogel such that it helps to prevent, reduce, or treat capsular contracture occurring in response to the implantation of breast prostheses in addition to supporting the weight of the breasts. The bras can be of any conventional design available in the art and be constructed of conventional materials such as cotton, LYCRA, polyester, or blends thereof.

In another aspect, the drug delivery device is in the form of a drug delivery bra cushion for preventing, reducing, or treating capsular contracture occurring in response to the implantation of breast prostheses, the drug delivery bra cushion comprising a layer of silk fibroin hydrogel which releases a composition comprising a therapeutic compound selected from the group consisting of EP₄ receptor agonists, EP₂ receptor agonists, or a combination thereof, in an amount from about 0.0001 mg to about 2 mg/kg/day. The drug delivery bra cushions can be inserted into bras that mammoplasty patients wear post-surgery. The drug delivery bra cushions can be of any design for conventional bra cushions and be constructed of conventional materials used for bra cushions.

What is claimed is:

1. An implantable prosthesis for preventing, reducing, or treating capsular contracture, the prosthesis comprising an inflatable envelope, a fillable cavity enclosed by the envelope, and a structure coupled to the envelope and effective to release a composition comprising a therapeutic compound, the compound being selected from the group consisting of EP₄ receptor agonists, EP₂ receptor agonists, or a combination thereof, in an amount from about 0.0001 mg to about 2 mg/kg/day, for between about two months and about one year.

2. The prosthesis of claim 1 wherein the structure comprises at least one osmotic pump coupled to the inflatable envelope.

3. The prosthesis of claim 1 wherein the structure comprises a coating on the inflatable envelope.

4. The prosthesis of claim 1 wherein the structure comprises a silk fibroin hydrogel coating.

5. The prosthesis of claim 1 wherein the compound comprises a therapeutically effective amount of a compound of Formula (I) or a pharmaceutically acceptable salt 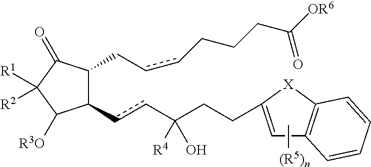
wherein each dashed line represents the presence or absence of a double bond;
R¹, R², R³ and R⁴ are each independently selected from H and C₁-C₆ alkyl;
R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O; wherein said compound is present alone or in combination with one or more pharmaceutically acceptable excipients.

6. The prosthesis of claim 1 wherein the composition comprises a therapeutically effective amount of Compound I having the structure: 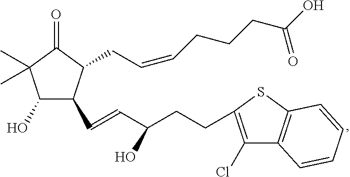
wherein said compound is present alone or in combination with one or more pharmaceutically acceptable excipients.

7. The prosthesis of claim 1 wherein the compound comprises a therapeutically effective amount of Compound II having the structure: 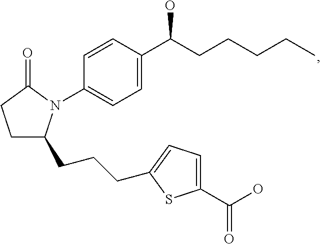
wherein said compound is present alone or in combination with one or more pharmaceutically acceptable excipients.

8. A method for preventing and treating capsular contracture occurring in response to the implantation of a prosthesis, the method comprising administering a composition comprising a therapeutic compound selected from the group consisting of EP4 receptor agonists, EP2 receptor agonists, or a combination thereof, to the implantation site of a patient, in an amount from about 0.0001 mg to about 2 mg/kg/day.

9. The method of claim 8, wherein the prosthesis is a breast prosthesis and the patient is undergoing mammoplasty.

10. The method of claim 8 wherein the administration is to dissection sites on a breast during the dissection for pocket development in a patient undergoing mammoplasty.

11. The method of claim 8 wherein the administration takes place prior to the first incision.

12. The method of claim 8 wherein the administration is to sites of incision and dissection made on a breast in order to remove a tissue expander.

13. The method of claim 8, wherein the therapeutic compound is a compound of Formula (I): 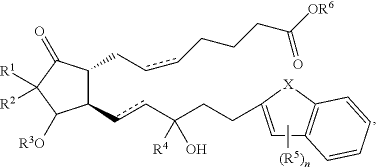
wherein each dashed line represents the presence or absence of a double bond; R¹, R², R³ and R⁴ are each independently selected from H and C₁-C₆ alkyl;
R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O.

14. The method of claim 8, wherein the therapeutic compound is Compound I:

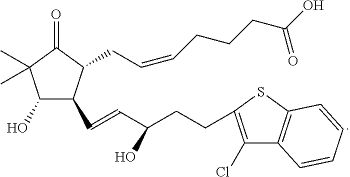

15. The method of claim 8, wherein the therapeutic compound is Compound II:

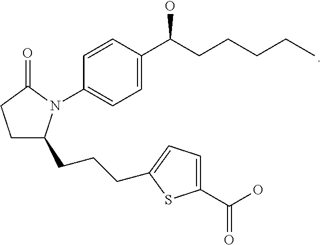

16. An implantable tissue expander for preventing, reducing, or treating capsular contracture occurring in response to the implantation of breast prostheses, the tissue expander comprising an inflatable envelope, a fillable cavity enclosed by the envelope, and silk fibroin hydrogel coating which releases a composition comprising a therapeutic compound selected from the group consisting of EP₄ receptor agonists, EP₂ receptor agonists, or a combination thereof, in an amount from about 0.0001 mg to about 2 mg/kg/day, for the duration of the tissue expander's implantation in the patient.

17. The tissue expander of claim 16, wherein the duration of the tissue expander's implantation in the patient is between about two months and about one year.

18. The tissue expander of claim 16, wherein the therapeutic compound is a compound of Formula (I): 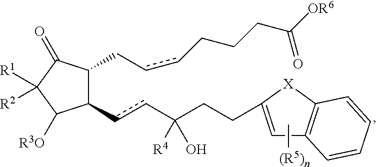
wherein each dashed line represents the presence or absence of a double bond;
R¹, R², R³ and R⁴ are each independently selected from H and C₁-C₆ alkyl;
R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O.

19. The tissue expander of claim 16, wherein the therapeutic compound is Compound I:

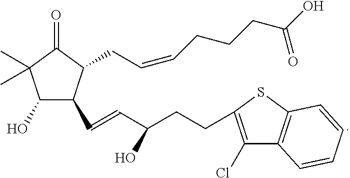

20. The tissue expander of claim 16, wherein the therapeutic compound is Compound II:

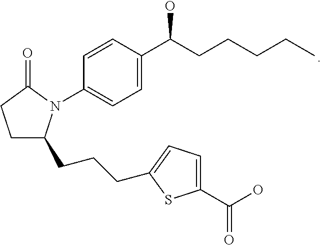

21. Medical dressing comprising silk fibroin hydrogel which releases a composition comprising a therapeutic compound selected from the group consisting of EP₄ receptor agonists, EP₂ receptor agonists, or a combination thereof, in an amount from about 0.0001 to about 2 mg/kg/day, for about one to about ten days.

22. The medical dressing of claim 21, wherein the therapeutic compound is a compound of Formula (I): 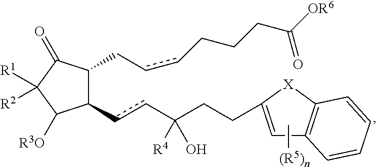
wherein each dashed line represents the presence or absence of a double bond;
R¹, R², R³ and R⁴ are each independently selected from H and C₁-C₆ alkyl;
R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O.

23. The medical dressing of claim 21, wherein the therapeutic compound is Compound I:

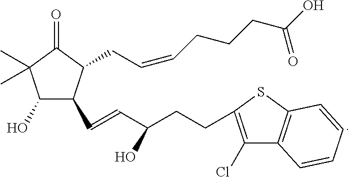

24. The medical dressing of claim 21, wherein the therapeutic compound is Compound II:

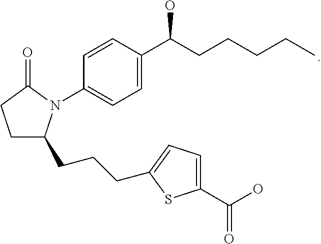

25. A drug delivery device for preventing, reducing, or treating capsular contracture occurring in response to the implantation of breast prostheses, the drug delivery device comprising a at least one breast cup, having a layer of silk fibroin hydrogel, for contacting skin when the cup is worn on a breast, the hydrogel capable of releasing a composition comprising a therapeutic compound selected from the group consisting of EP₄ receptor agonists, EP₂ receptor agonists, or a combination thereof, in an amount from about 0.0001 mg to about 2 mg/kg/day.

26. The drug delivery device of claim 25, wherein the therapeutic compound is a compound of Formula (I): 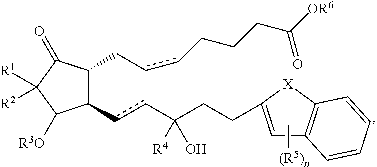
wherein each dashed line represents the presence or absence of a double bond;
R¹, R², R³ and R⁴ are each independently selected from H and C₁-C₆ alkyl;
R⁵ is halogen, C₁-C₆ alkyl, or C₂-C₆ alkenyl; R⁶ is H, C₁-C₆ alkyl, C₂-C₆ alkenyl, a salt thereof, or an amine thereof; n is 0-7; and X is S or O.

27. The drug delivery device of claim 25, wherein the therapeutic compound is Compound I:

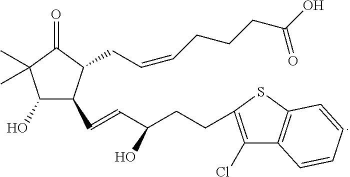

28. The drug delivery device of claim 25, wherein the therapeutic compound is Compound II: [chemical structure]
